(19) United States
(12) Patent Application Publication
(10) Pub. No.: US 2020/0054046 A1
(43) Pub. Date: Feb. 20, 2020

(54) THERMALLY PROCESSING FOOD PRODUCTS WITH HIGHLY-UNIFORM ELECTROMAGNETIC ENERGY FIELDS

(71) Applicant: Campbell Soup Company, Camden, NJ (US)

(72) Inventors: Scott Wayne Keller, Medford, NJ (US); Jungang Wang, Collingswood, NJ (US); Mark Robert Watts, Marlton, NJ (US); Mark Firary, Philadelphia, PA (US); Rasheed Mohammed, Sicklerville, NJ (US)

(21) Appl. No.: 16/543,097

(22) Filed: Aug. 16, 2019

Related U.S. Application Data
(60) Provisional application No. 62/719,294, filed on Aug. 17, 2018.

(51) Int. Cl. A23L 3/01; H05B 6/78

(52) U.S. Cl. CPC A23L 3/01; A61L 2/04; H05B 6/782

(57) ABSTRACT

Embodiments herein relate to systems and methods for thermally processing packaged food products with highly-uniform electromagnetic energy fields. In an embodiment, a method of thermally processing a packaged food product is included herein. The method can include placing the packaged food product in a heating chamber, the packaged food product comprising a hermetically sealed package and a food material disposed within the package. The method can further include applying electromagnetic energy to the packaged food product from a first electromagnetic energy source from a first direction. The electromagnetic energy can be sufficient to raise the temperature of the food material by at least 50 degrees Fahrenheit with a spatial temperature variation of less than 25 degrees Fahrenheit in less than 120 seconds. Other embodiments are also included herein.

This application claims the benefit of U.S. Provisional Application No. 62/719,294, filed Aug. 17, 2018, the content of which is herein incorporated by reference in its entirety.

### FIELD

Embodiments herein relate to systems and methods for thermally processing packaged food products with highly-uniform electromagnetic energy fields.

### BACKGROUND

Most food products have a tendency to spoil relatively quickly. As such, preservation techniques have been developed over many years to extend the amount of time that a given food product will remain fresh. Food preservation techniques can include dehydrating, freezing, fermenting, pickling, acidification, curing, canning, heat treating, retort sterilization, irradiating, chemical preservation and the like.

Traditional retort sterilization typically involves the application of heat to hermetically sealed packages of food through thermal conduction. Retort sterilization allows for packaged non-frozen and non-dehydrated ready-to-eat foods that can have a shelf life of months to years.

While food preservation techniques, such as retort sterilization, have been successful at preventing food spoilage, it has been found that such techniques can have adverse effects on food products including, diminishing taste and appearance, reducing nutritional qualities, and the like.

### SUMMARY

Embodiments herein relate to systems and methods for thermally processing packaged food products with highly-uniform electromagnetic energy fields. In an embodiment, a method of thermally processing a packaged food product is included herein. The method can include placing the packaged food product in a heating chamber, the packaged food product comprising a hermetically sealed package and a food material disposed within the package. The method can further include applying electromagnetic energy to the packaged food product from a first electromagnetic energy source from a first direction. The electromagnetic energy can be sufficient to raise the temperature of the food material by at least 50 degrees Fahrenheit with a spatial temperature variation of less than 25 degrees Fahrenheit in less than 120 seconds.

In an embodiment, a method of thermally processing a packaged food product is included. The method can include placing the packaged food product in an environment with a pressure greater than 0 psig and applying electromagnetic energy to the packaged food product from a first electromagnetic energy source from a first direction. The electromagnetic energy can be applied at least 50 percent of the time during a 180 second period and the average temperature of the food material can be increased by at least 100 degrees Fahrenheit while no portion of the food material reaches a temperature exceeding 265 degrees Fahrenheit sustained for at least 5 seconds.

In an embodiment, a method of thermally processing a packaged food product is included. The method can include placing the packaged food product in an environment with a pressure greater than 0 psig. The method can also include applying electromagnetic energy to the packaged food product from a first electromagnetic energy source from a first direction. The temperature can be raised to at least 100 degrees Fahrenheit in 180 seconds or less and at least 40 percent of the energy required to raise the temperature can be provided by electromagnetic energy absorbed by portions of the packaged food product.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of phases of thermally processing packaged food products in accordance with various embodiments herein.

FIG. 2 is a schematic side view of a processing system in accordance with various embodiments herein.

FIG. 3 is a schematic perspective view of a packaged food product in accordance with various embodiments herein.

FIG. 4 is a schematic cross-sectional view of the packaged food product of FIG. 3 as taken along line 4-4′ of FIG. 3 in accordance with various embodiments herein.

FIG. 5 is a cross-sectional thermal view of the packaged food product of FIGS. 3 and 4 as taken along line 5-5′ of FIG. 4 resulting from thermal processing with a non-uniform electromagnetic field.

FIG. 6 is a graph showing temperature over time corresponding with heating operations during thermal processing of a packaged food product using a non-uniform electromagnetic field.

FIG. 7 is a cross-sectional thermal view of the packaged food product of FIGS. 3 and 4 as taken along line 5-5′ of FIG. 4 resulting from thermal processing with a highly-uniform electromagnetic field in accordance with various embodiments herein.

FIG. 8 is graph showing temperature over time corresponding with heating operations during thermal processing of a packaged food product using a highly-uniform electromagnetic field in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Electromagnetic fields with waves at specific frequencies such microwaves have proven to be very useful in food processing applications. For example, in consumer microwave ovens, microwave energy can be used to efficient heat up food to serving temperatures. In some cases, microwaves have also been applied to heat up food product to assist in sterilization and/or pasteurization processes.

It has been found that electromagnetic energy sources, such as microwave and/or radiofrequency energy sources, create a field that is substantially non-uniform in its spatial field strength. As a result, the application of such energy at high levels creates an uneven temperature distribution throughout the food product to which the energy is applied. This can be problematic as overheated food may have undesirable taste, texture, nutritional properties and the like. In addition, significant overheating can potentially damage the packaging in which the food material of the food product is contained. As such, many systems must purposefully limit the intensity and the duration of the energy applied and then allow for periods of temperature equilibration in the absence of intense microwave and/or radiofrequency wave energy application to allow for heat to move from hot spots to cooler or cold spots primarily through thermal conduction.

However, embodiments herein include a method of thermally processing a packaged food product with a highly-uniform electromagnetic energy field. Specifically, in some embodiments, a method of sterilizing or pasteurizing a packaged food product with a highly-uniform electromagnetic energy field is included. The use of a highly-uniform electromagnetic energy field provides various advantages. As one example, a highly-uniform electromagnetic energy field (e.g., microwave and/or radiofrequency wave) can be applied at a high level of intensity for a period of time to quickly raise the temperature of a food product without the spatial temperature variation (e.g., a temperature delta between hot spots and cold spots) that is typically associated with heating techniques such as microwave heating. Further, because the temperature delta between hot spots and cold spots is greatly reduced, the need to allow time for temperature equilibration through thermal conduction is also greatly reduced. Therefore, the percent of the time during the overall heating process during which microwaves and/or radiofrequency waves can be applied can be greatly increased.

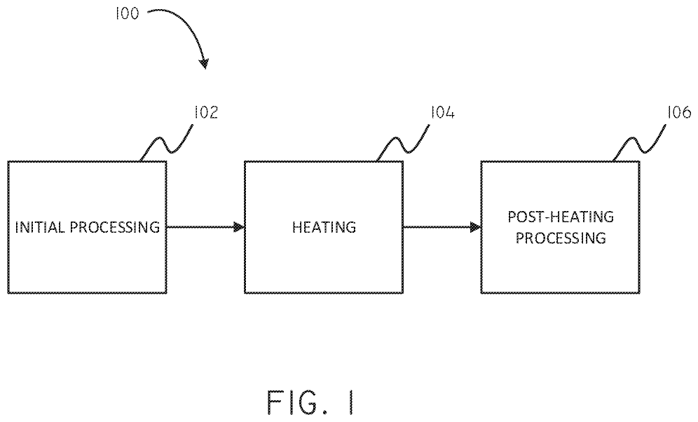

Referring now to FIG. 1, a schematic view is shown of phases of thermally processing packaged food products in accordance with various embodiments herein. A process of thermally processing packaged food products can include phases of initial processing 102, a heating phase 104, and post-heating processing 106. It will be appreciated that each of these phases can include multiple steps. By way of example, the initial processing phase 102 can include steps of pressurizing, preheating, placing of packaged food products in trays or carriers, immersing the packaged food products in a fluid (such as a liquid including, but not limited to, water, or a gas), etc. Further, the heating phase 104 can include steps of applying electromagnetic wave energy (such as microwave and/or radiofrequency waves), providing temperature equilibration time while electromagnetic wave energy is withheld or substantially attenuated, holding the packaged food product at a target temperature for a specific amount of time (e.g., a “hold” step or phase), and the like. The post-heating processing phase 106 can include steps of cooling the packaged food product down, depressurizing the packaged food product, removing packaged food products from trays or carriers, stacking packaged food products, and the like. It will be appreciated that the foregoing only serves as examples of steps that can be performed and that additional steps or fewer steps can be performed at each phase in various embodiments.

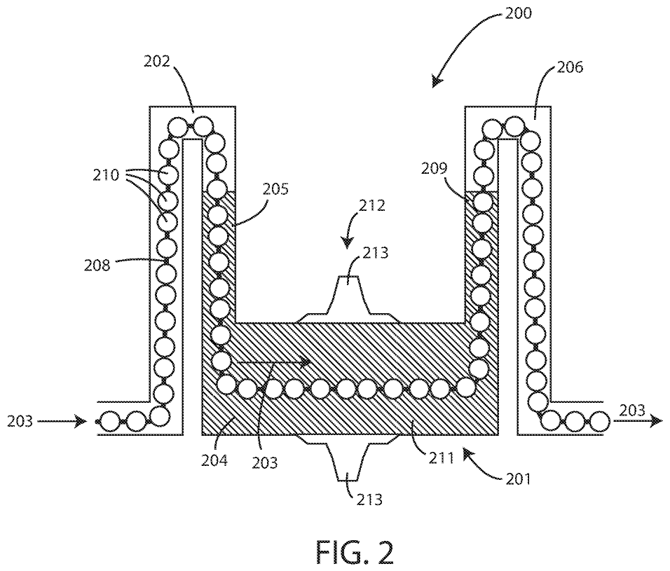

Embodiments herein can be performed with various pieces of equipment. Referring now to FIG. 2, a schematic side view is shown of a processing system in accordance with various embodiments herein. The processing system 200 includes a continuous processing channel 201. The continuous processing channel 201 can include a pressurizing chamber or zone 202. In some embodiments, the pressurizing chamber can include increasing the pressure to which the food products are exposed. In some embodiments, the pressurizing chamber can also include the initial application of heat to food products and thereby raising the temperature of the food products. However, in other embodiments, substantially no heat is applied to the food products in the pressurizing chamber. The continuous processing channel can also include a heating chamber or zone 204 and a cool-down chamber or zone 206 (or in cases where cooling is not done at this stage an output chamber).

In some embodiments, the pressurizing chamber 202 can be oriented for vertical product movement. In specific, the pressurizing chamber 202 can be oriented for vertical movement of food products (or trays or flights of food products) 210 along a product conveyor mechanism 208 through the continuous processing channel 201 of the processing system 200 in the direction of arrows 203. In some embodiments, an actuator or similar mechanism can be disposed within the pressurizing chamber 202 in order to cause rotation (such as axial rotation) of the food products.

Various mechanisms can be used to begin warming the food products within the pressurizing chamber 202. By way of example, a microwave emitter array can be positioned to begin heating products within the pressurizing chamber 202. In some embodiments, the fluid within the pressurizing chamber 202 can itself be heated in order to transfer heat to the food products through conduction. However, in various embodiments herein, very little or no heating of the food products is performed within the pressurizing chamber 202.

The pressurizing chamber 202 can include a fluid column 205. In this case, the fluid column 205 is in fluid communication with the microwave or radiofrequency heating chamber 204. The fluid column 205 exerts a force downward onto the fluid in the microwave or radiofrequency heating chamber 204 such that the pressure in the microwave or radiofrequency heating chamber 204 is higher than in the area above the fluid column 205 (for example, in many cases above atmospheric pressure). In some embodiments, the maximum pressure within the pressurizing chamber 202 is from about 0 psig to about 60 psig. In some embodiments, the temperature of the fluid in the pressurizing chamber 202 can be from about 32 degrees Fahrenheit to about 300 degrees Fahrenheit.

The height of the pressurizing chamber 202 can vary. In general, the taller the pressurizing chamber is, the taller the water column(s) therein can be. As such, the height can vary depending on the desired water column height which in turn can vary based on desired pressures. However, in some embodiments the height of the pressurizing chamber can be greater than about 2, 4, 6, 8, 10, 15, 20, 25, 30, 40, 50, 60, 70, or 100 feet. In some embodiments, the height of the pressurizing chamber can be in a range wherein each of the foregoing numbers can serve as the lower or upper bound of the range provided that the upper bound is higher than the lower bound.

In some embodiments the height of one or more water columns in the pressurizing chamber can be greater than about 1, 3, 5, 7, 9, 14, 19, 24, 29, 39, 49, 59, 69, or 99 feet. In some embodiments, the height of one or more water columns in the pressurizing chamber can be in a range wherein each of the foregoing numbers can serve as the lower or upper bound of the range provided that the upper bound is higher than the lower bound.

In some embodiments, the pressurizing chamber 202 can be substantially air-tight except for the area where food products enter the pressurizing chamber 202 and the area where food products exit the pressurizing chamber 202. In some embodiments, access hatches or ports (including but not limited to fluid exchange ports) and/or observation windows can be included at various points along the path of the pressurizing chamber 202.

Food products 210 can be moved by the product conveyor mechanism 208 from the pressurizing chamber 202 and into a following chamber such as the microwave or radiofrequency heating chamber 204. It will be appreciated, however, that in some embodiments food products may enter a holding chamber before entering the microwave or radiofrequency heating chamber 204. The microwave or radiofrequency heating chamber 204 can be filled with a fluid 211.

The processing system 200 can include a microwave energy emitting apparatus 212 in order to deliver microwave energy to the microwave or radiofrequency heating chamber 204. In some embodiments, an actuator or similar mechanism can be disposed within the microwave or radiofrequency heating chamber 204 in order to cause rotation (such as axial rotation) of the food products. However, in other embodiments, the conveyor mechanism 208 in the microwave or radiofrequency heating chamber 204 is designed to hold the food products in a substantially static plane.

In some embodiments, the head space above the food products in the microwave or radiofrequency heating chamber 204 (e.g., distance between the top of the food product and the inner wall of the microwave or radiofrequency heating chamber above the food product) is relatively small. By way of example, the head space can be less than about 50 cm, 40 cm, 30 cm, 20 cm, 10 cm, 5 cm, or 1 cm. In some embodiments, the head space can be greater than about 0.2 cm, 0.5 cm, 0.8 cm, 1 cm, 1.5 cm, 2 cm, 3 cm, or 5 cm. In some embodiments, the head space can be in a range with any of the preceding numbers representing the lower and upper bounds of the range provided that the upper bound is larger than the lower bound.

In some embodiments, the microwave or radiofrequency heating chamber 204 can be substantially air-tight except for the area where food products enter the microwave or radiofrequency heating chamber 204 and the area where food products exit the microwave or radiofrequency heating chamber 204. In some embodiments, access hatches or ports (including but not limited to fluid exchange ports) and/or observation windows can be included at various points along the path of the microwave or radiofrequency heating chamber 204.

In some embodiments, the temperature of the fluid in the microwave or radiofrequency heating chamber 204 can be from about 32 degrees Fahrenheit to about 300 degrees Fahrenheit. In some embodiments, the fluid temperature can be stabilized to a target temperature using a heat exchanger, heat regulator, heating device, cooling device, etc.

The microwave energy emitting apparatus 212 can include one or more microwave units 213. In some embodiments, each microwave unit 213 can be separate from one another and can each have their own emitter (such as a magnetron or other emitter), waveguide, horn, waveguide cover, slotted waveguide, etc. In other embodiments, microwave units 213 can share components such as a shared magnetron. In some embodiments, the microwave units 213 can be arranged into an array. By way of example, in some embodiments, the microwave energy emitting apparatus 212 can include from 1 to 40 microwave units 213. In some embodiments, the microwave units 213 can be arranged into a grid.

In some embodiments, the microwave units can be placed at varied distances from each other to allow food product within each food package to equilibrate in temperature before traveling under the next microwave unit. In contexts where it is relevant, the equilibrium period could range from 1 second to 20 minutes. In some embodiments, the speed of the conveyor mechanism can be changed to accommodate a desired thermal equilibration time. By way of example, in some embodiments, the conveyor mechanism can be stopped or slowed down to accommodate a desired thermal equilibration time.

In some embodiments, the microwave energy emitting apparatus 212 can be configured to emit energy continuously. In some embodiments, the microwave energy emitting apparatus 212 can be configured to emit energy intermittently. In some embodiments, the intensity of the emitted energy can be constant. In some embodiments, the intensity of the emitted energy can be varied. In some embodiments, the microwave energy emitting apparatus 212 can be configured to emit energy in response to one or more triggering events, such as when food products pass a triggering sensor.

In some embodiments, the microwave units 213 can emit microwave energy at a frequency from approximately 300 MHz to approximately 2550 MHz or between 800 MHz to approximately 2550 MHz. In some embodiments, the microwave units 213 can emit microwave energy at a frequency from approximately 915 MHz or approximately 2450 Mhz. In some embodiments, all microwave units 213 can emit microwave energy at a common frequency. In other embodiments, microwave units 213 can emit energy at different frequencies. For example, the microwave units 213 can emit microwave energy at a first frequency of approximately 915 MHz and a second frequency of approximately 2450 Mhz. It is believed that higher frequencies, such as around 2450 MHz, can be useful for surface related effects such as browning, searing, carmelization, etc. In some embodiments, units emitting at higher frequencies around 2450 MHz can be disposed toward the end of the microwave or radiofrequency heating chamber. In some embodiments, other types of heating units that may be useful in browning or similar processes, such as infrared heating units, can be preferentially disposed toward the end of the microwave or radiofrequency heating chamber.

While in many embodiments the system can include the application of microwave energy, in other embodiments, energy can be applied from another portion of the electromagnetic spectrum, either by itself or in combination with other wavelengths of electromagnetic radiation. For example, in various embodiments herein, the application of electromagnetic energy with a frequency of between 13.56 MHz to 300 MHz can be included. It will be appreciated that references herein to chambers of the apparatus, emitters, and other components that specifically reference microwaves are also applicable in the context of the application of electromagnetic radiation with a frequency of between about 13.56 MHz to about 300 MHz.

In general, microwave energy at lower frequencies (e.g., around 915 MHz) penetrate into food products more deeply than microwave energy at a higher frequency (e.g., around 2450 MHz). In some embodiments, emitters that provide microwave energy at frequencies that penetrate less (e.g., higher frequencies) can be arranged toward the downstream side of the microwave or radiofrequency heating chamber 204 and thus closer in both proximity and time to the cool-down chamber 206. Similarly, emitters that provide microwave energy at frequencies that penetrate more (e.g., lower frequencies) can be arranged toward the upstream side of the microwave or radiofrequency heating chamber 204 to accommodate the placement of the other emitters.

While the microwave units 213 in FIG. 2 are shown arranged on the top and bottom of the microwave or radiofrequency heating chamber 204, it will be appreciated that the microwave units 213, or at least a portion of them such as a waveguide, horn, waveguide cover, slotted waveguide, or the like can be arranged on any of the top, bottom, or sides of the microwave or radiofrequency heating chamber 204. In some embodiments the microwave units 213 are arranged opposed from one another on opposite sides of the microwave or radiofrequency heating chamber 204. In some embodiments, microwave units 213 can be arranged in an offset or staggered pattern.

The microwave units 213 and/or the system can be configured to deliver electromagnetic energy to the food packages multidirectionally or unidirectionally. In many embodiments, the microwave units 213 and/or the system can be configured to deliver electromagnetic energy to the food packages unidirectionally. As such, in embodiments providing electromagnetic energy unidirectionally, the system herein stands in contrast to many consumer microwave ovens wherein electromagnetic energy bounces off walls and may therefore hit an item to be heated from many different angles simultaneously. In various embodiments, stray electromagnetic energy can be absorbed by the fluid in the system surrounding the food products. In some embodiments, the interior of one or more chambers of the system can be lined with a material that absorbs electromagnetic energy instead of reflecting it.

Food products 210 can be moved by the product conveyor mechanism 208 from the microwave or radiofrequency heating chamber 204 and into a following chamber such as the cool-down chamber 206. It will be appreciated, however, that in some embodiments food products may enter a holding chamber before entering the cool down chamber 206.

The cool-down chamber 206 can also be oriented for vertical product movement. In specific, the cool-down chamber 206 can be oriented for vertical movement of food products 210 (or a flight of food products) along a product conveyor mechanism 208 through the continuous processing channel 201 of the processing system 200 in the direction of arrows 203. In some embodiments, an actuator or similar mechanism can be disposed within the cool-down chamber 206 in order to cause rotation (such as axial rotation) of the food products.

The cool-down chamber 206 can also include a fluid column 209. In this case, the fluid column 209 is in fluid communication with the microwave or radiofrequency heating chamber 204. The fluid column 209 exerts a force downward onto the fluid in the microwave or radiofrequency heating chamber 204 such that the pressure in the microwave or radiofrequency heating chamber 204 is higher than in the area above the fluid column 209 (for example, in many cases above atmospheric pressure). In some embodiments, the maximum pressure within the cool-down chamber 206 is from about 0 psig to about 60 psig. In various embodiments, the temperature of the fluid in the cool-down chamber 206 can be from about 32 degrees Fahrenheit to about 300 degrees Fahrenheit. The final temperature of food products exiting the system can vary, but in some embodiments the final temperature (exit temperature) can be from about 32 degrees to about 212 degrees. In some embodiments the final temperature (exit temperature) can be from about 80 degrees to about 150 degrees.

In some embodiments, the cool-down chamber 206 can be substantially air-tight except for the area where food products enter the cool-down chamber 206 and the area where food products exit the cool-down chamber 206. In some embodiments, access hatches or ports (including but not limited to fluid exchange ports) and/or observation windows can be included at various points along the path of the cool-down chamber 206.

The height of the cool-down chamber 206 can vary. In general, the taller the cool-down chamber is, the taller the water column(s) therein can be. As such, the height can vary depending on the desired water column height which in turn can vary based on desired pressures. However, in some embodiments the height of the cool-down chamber can be greater than about 2, 4, 6, 8, 10, 15, 20, 25, 30, 40, 50, 60, 70, or 100 feet. In some embodiments, the height of the cool-down chamber can be in a range wherein each of the foregoing numbers can serve as the lower or upper bound of the range provided that the upper bound is higher than the lower bound.

In some embodiments the height of one or more water columns in the cool-down chamber can be greater than about 1, 3, 5, 7, 9, 14, 19, 24, 29, 39, 49, 59, 69, or 99 feet. In some embodiments, the height of one or more water columns in the cool-down chamber can be in a range wherein each of the foregoing numbers can serve as the lower or upper bound of the range provided that the upper bound is higher than the lower bound.

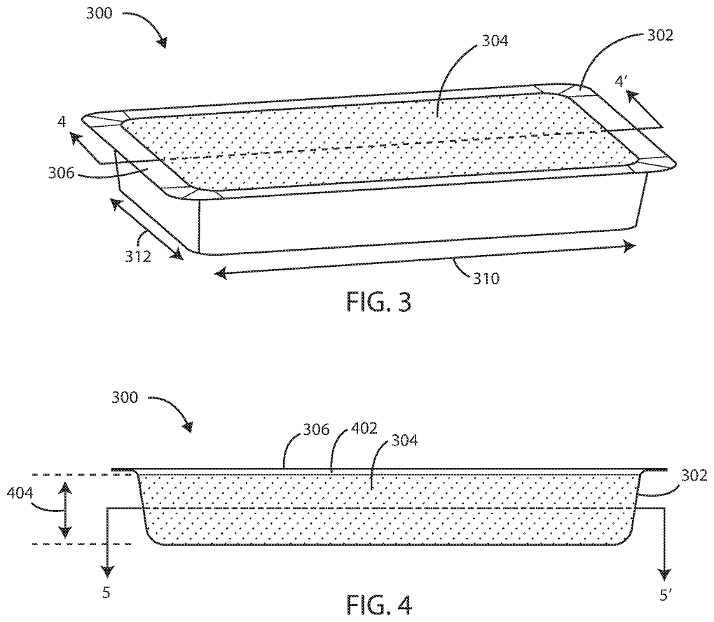

Referring now to FIG. 3, a schematic perspective view is shown of a packaged food product 300 in accordance with various embodiments herein. The packaged food product 300 can include a food material 304 disposed within a package 302, such as a tray. The packaged food product 300 can have a length 310 and a width 312. The length 310 and the width 312 can vary. In some embodiments, the length 310 can be about 3 to 30 cm. In some embodiments, the width 312 can be about 2 to 25 cm. A sealing film 306 can be disposed over the food material 304 and sealed to the top of the package 302 creating a hermetic seal.

Referring now to FIG. 4, a schematic cross-sectional view is shown of the packaged food product 300 of FIG. 3 as taken along line 4-4′ of FIG. 3 in accordance with various embodiments herein. The package 302 can define a space into which the food material 304 is disposed. In some embodiments, there can be a head space 402 between the sealing film 306 and the top of the food material 304. The food material 304 within the package 302 can have a height 404. The height 404 can vary, but in some embodiments can be about 0.5, 1, 1.5, 2, 2.5, 3, 3.5, 4, 5, 6, 7, 8, 9, 10, 11, 12 13, 14, or 15 centimeters or can fall within a range between any of the foregoing.

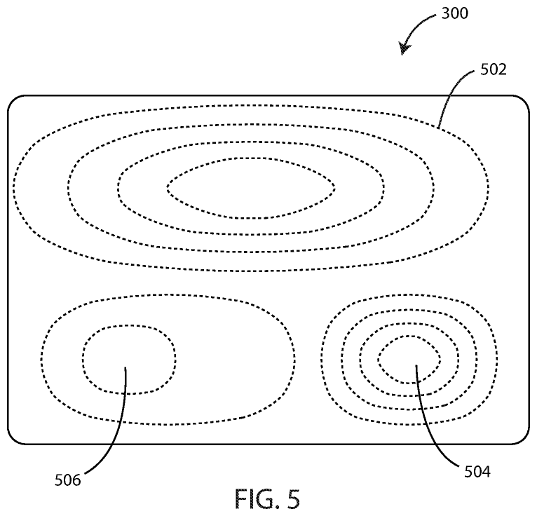

Referring now to FIG. 5, a cross-sectional thermal view is shown of the packaged food product of FIGS. 3 and 4 as taken along line 5-5′ of FIG. 4 resulting from thermal processing with a non-uniform electromagnetic field. In specific, this is an idealized example illustrating the substantial spatial temperature variation resulting from heating with radiation from a non-uniform electromagnetic wave emitter (such as a microwave or radiofrequency emitter). This thermal view shows thermal contour lines 502. It also illustrates a hot spot 504 (the hottest area within the food material 304) and a cold spot 506 (the coldest area within the food material 304). The temperature delta in this example is the difference between the temperature at the hot spot 504 and at the cold spot 506. As can be seen by the number of thermal contour lines between the hot spot 504 and the cold spot 506, there is a substantial temperature delta illustrated between the hot spot 504 and the cold spot 506. For example, if each thermal contour line marks temperature in Fahrenheit by 10 s, then the temperature delta between the hot spot 504 and the cold spot 506 in this example is on the order of 60 degrees Fahrenheit.

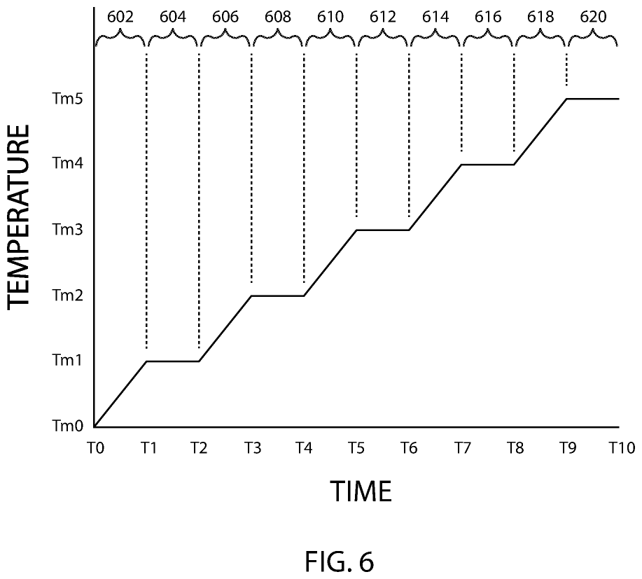

The significant temperature delta illustrated with respect to FIG. 5 can be problematic for various reasons including requiring a process with multiple phases wherein the application of microwave energy is ceased or substantially attenuated and the temperature within the package is allowed to equilibrate largely through thermal conduction. Referring now to FIG. 6, a graph is shown illustrating temperature over time corresponding with heating subphases during thermal processing of a packaged food product using a non-uniform electromagnetic field. In this view, it can be seen that the temperature within a particular spot in the food material goes up in a step-like fashion from a starting temperature Tm0 at time T0 to an ending temperature Tm5 at time T9. During “on” subphases 602, 606, 610, 614, and 618 electromagnetic wave energy (microwave or radiofrequency wave) is applied and the temperature rises quickly. However, during “off” subphases 604, 608, 612, 616 and 620, the application of electromagnetic wave energy is ceased or substantially attenuated. As such, the temperature during these phases remains relatively constant (this assumes that the spot measured is already at an equilibration temperature and is not disposed at or adjacent to a hot or cold spot) or may gradually rise if the environment surrounding the package food product in the system is warmer than the packaged food product. Regardless, the temperature change during the “off” phases is much less than during the “on” phases. Because of the large temperature delta between the hot spot and the cold spot illustrated with regard to FIG. 5, this “on” and “off” cycling is necessary to allow for temperature equilibration and prevent damage to the food material and the packaging. As a result, the total amount of time for the temperature of the food material to reach Tm5 is the difference between time T9 and time T0, which can be quite significant depending on the absolute temperature value of Tm5.

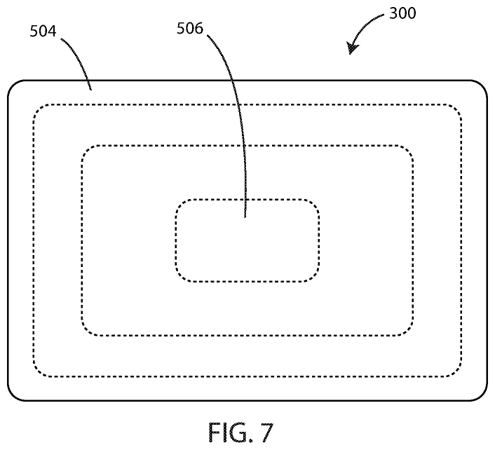

In contrast to FIG. 5, highly-uniform electromagnetic energy fields herein can be used to provide much more rapid and even heating of food materials. Referring now to FIG. 7, a cross-sectional thermal view is shown of the packaged food product of FIGS. 3 and 4 as taken along line 5-5′ of FIG. 4 resulting from thermal processing with a highly-uniform electromagnetic field in accordance with various embodiments herein. The temperature delta in this example is the difference between the temperature at the hot spot 504 and at the cold spot 506. As can be seen by the number of thermal contour lines between the hot spot 504 and the cold spot 506, there is a temperature delta illustrated between the hot spot 504 and the cold spot 506 that is much less than the temperature delta illustrated in FIG. 5. For example, if each thermal contour line marks temperature in Fahrenheit by 10 s, then the temperature delta between the hot spot 504 and the cold spot 506 in this example is on the order of 30 degrees Fahrenheit.

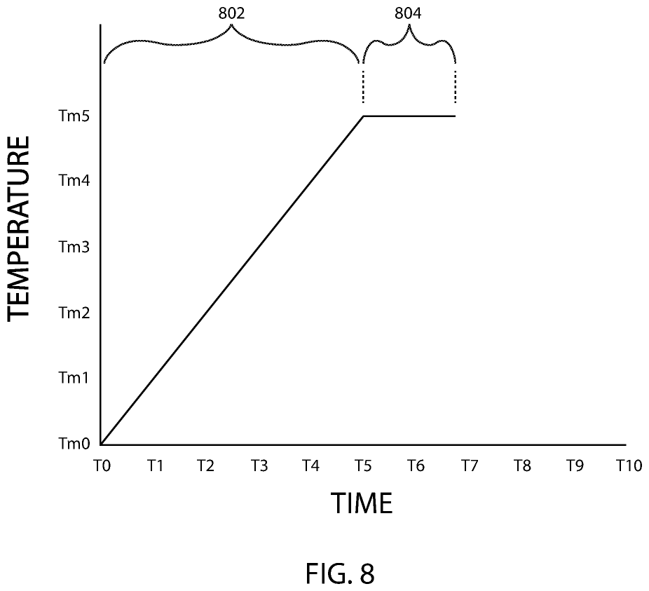

The greatly reduced temperature delta illustrated in FIG. 7 can allow for a process wherein the application of microwave energy can be much more continuously applied in order to raise the temperature of the food material more quickly. As such, much less time, if any, must be devoted to ceasing or substantially attenuating the microwave or radiofrequency energy and allowing the temperature within the package to equilibrate largely through thermal conduction. Referring now to FIG. 8, a graph is shown illustrating temperature over time corresponding with heating operations during thermal processing of a packaged food product using a highly-uniform electromagnetic field in accordance with various embodiments herein. In this view, it can be seen that the temperature within a particular spot in the food material goes up in a much more continuous and rapid fashion compared with FIG. 6 from a starting temperature Tm0 at time T0 to an ending temperature Tm5 at time T5. In this view, there is a single “on” subphase 802 wherein electromagnetic wave energy (microwave or radiofrequency wave) is applied and the temperature rises quickly. After the “on” phase, there is an “off” subphase 804, wherein the application of electromagnetic wave energy is ceased or substantially attenuated, which can allow for a degree of temperature equilibration and/or provide for a “hold” at a desired treatment temperature. Because of the much smaller temperature delta between the hot spot and the cold spot illustrated with regard to FIG. 7 (in contrast to FIG. 5), much less “on” and “off” cycling is necessary and the total amount of time for the temperature of the food material to reach Tm5 is the difference between time T5 and time T0, which can be significantly less than as illustrated with regard to FIG. 5. It will be appreciated that this is an idealized example and that in some embodiments, there still may be one or more “off” subphases that interrupt the “on” subphase (resulting in multiple “on” phases), but in any regard this can be markedly reduced compared to the type of scenario illustrated with regard to FIG. 5. Also, it will be appreciated that because of various factors including, but not limited to, how dielectric factors can change with temperature and the impact of some amount of heat conduction, and the like, that an actual temperature/time curve is likely to appear less linear through the various phases. Regardless, it will be appreciated that FIGS. 5-8 clearly illustrate the principles to one of skill in the art.

In some embodiments, “off” subphases can be executed through turning off an electromagnetic energy source. In some embodiments, “off” subphases can be executed through shielding an electromagnetic energy source. In some embodiments, “off” subphases can be executed by substantially attenuating an electromagnetic energy source (such as reducing the field strength by at least 40, 50, 60, 70, 80, 90, 95, 98, 99, or 100 percent, or by an amount falling within a range between any of the foregoing). In some embodiments, “off” subphases can be executed by passing the packaged food product out of the path of the electromagnetic energy source (such as moving the packaged food product away from an energy source or a component thereof on a conveyor belt of the like).

### Methods

In various embodiments herein, a method of thermally processing a packaged food product is included. The method can include operations of placing the packaged food product in a heating chamber. The packaged food product can include a hermetically sealed package and a food material disposed within the package. The heating chamber can have a pressure greater than 0 psig. The method can also include an operation of applying microwave or radiofrequency energy to the packaged food product from a first microwave or radiofrequency energy source from a first direction. The microwave or radiofrequency energy can be sufficient to raise the temperature of the food material by at least 30, 40, 50, 60, 70, 80, 90, 100, 110, 120, 130, 140, 150, 160, 170, or 180 degrees Fahrenheit (or an amount falling within a range between any of the foregoing) with a spatial temperature variation of less than 60, 50, 40, 30, 25, 20, 15, 10 or 5 degrees Fahrenheit (or an amount falling within a range between any of the foregoing) in less than 420, 360, 300, 240, 180, 120, 90, 60, 30, or 15 seconds. In some embodiments, the microwave or radiofrequency energy can specifically be sufficient to raise the temperature of the food material by at least 50 degrees Fahrenheit with a spatial temperature variation of less than 15 degrees Fahrenheit in less than 120 seconds.

In various embodiments, the method can include applying microwave or radiofrequency energy to the packaged food product from a second microwave or radiofrequency energy source from a second direction, wherein the second direction is substantially opposite the first direction. In various embodiments, the packaged food can be immersed in a fluid within the heating chamber. The heating chamber can have a pressure of greater than 0, 10, 15, 20, 25, 30, 35, 40, 45, 50, 55, 60, 65, 70, or 80 psig (or falling within a range between any of the foregoing).

In various embodiments herein, less than 60, 50, 40, 30, 20, 10, 5, or 1% (or a percentage falling within a range between any of the foregoing) of the total energy required to raise the temperature of the food material to a preselected temperature is provided through conductive heat transfer from the fluid to the packaged food product. In various embodiments the preselected temperature can be a maximum attained processing temperature, a required lethality temperature, a targeted lethality temperature, or a targeted process temperature. In various embodiments, the preselected temperature can be 120, 130, 140, 150, 160, 170, 175, 180, 185, 190, 195, 200, 205, 210, 212, 215, 220, 225, 230, 240, 245, 250, 255, 260 or 265 degrees Fahrenheit (or can fall within a range between any of the foregoing).

In some embodiments, the spatial temperature variation is measured across a two-dimensional area of the food material at least 1, 2, 3, 4, 5, 6, 7, 8, 10, 15, or 20 (or a range between any of the foregoing) centimeters by 1, 2, 3, 4, 5, 6, 7, 8, 10, 15, or 20 (or a range between any of the foregoing) centimeters in size at a depth of at least 0.1, 0.2, 0.5, 0.75, 1, 1.5, 3, 5, 10, 15, 20, 25 to 30 (or a range between any of the foregoing) centimeters below a top surface of the food material within the packaged food product. In some embodiments, the spatial temperature variation can be measured at a depth representing a midpoint between a top surface of the food material and a bottom surface of the food material. In some embodiments, the spatial temperature variation is measured across a three-dimensional volume representing the entirety of the food material.

In some embodiments, the total time between the beginning of the application of microwave or radiofrequency energy and the beginning of a hold phase is less than 3600, 2400, 1200, 720, 600, 540, 360, 300, 280, 260, 240, 220, 200, 180, 160, 140, 120, 100, 80, 60, 50, 40, or 30 seconds (or an amount of time falling within a range between any of the foregoing).

In some embodiments, the microwave or radiofrequency energy is applied at least about 40, 50, 60, 70, 80, 85, 90, or 95 percent of the time (or a percentage falling within a range between any of the foregoing) during a 60, 70, 80, 90, 100, 120, 140, 160, 180, 200, 220, 240, 260, 280, 300, 360, 540, or 720 second period (or an amount of time falling within a range between any of the foregoing).

In some embodiments, the average temperature of the food material is raised to at least 50, 60, 70, 80, 90, 100, 110, 120, 130, 140, 150, 160, 170, 180, 190, 200, 210, 220, or 230 degrees Fahrenheit (or a temperature falling within a range between any of the foregoing) and at least 50, 55, 60, 65, 70, 75, 80, 85, 90, or 95 percent (or a percentage falling within a range between any of the foregoing) of the energy required to raise the temperature is provided by electromagnetic energy or radiofrequency radiation. In some embodiments, a method of thermally processing a packaged food product is included. The method can include placing the packaged food product in an environment with a pressure greater than 0 psig and applying electromagnetic energy to the packaged food product from a first electromagnetic energy source from a first direction. The electromagnetic energy can be applied at least 40, 50, 60, 70, 80, or 90 percent of the time during a 60, 90, 120, 150, 180, 210, 240, 270, 300, 330, 360, 540, 720, 900, or 1200 second period, wherein the average temperature of the food material is increased by at least 50, 60, 70, 80, 90, 100, 110, 120, 130, 140, 150, 160, 170, 180, 190, 200, 210, 220, 230, 240 degrees Fahrenheit and no portion of the food material reaches a temperature exceeding 210, 220, 230, 240, 250, 260, 265, 270, 275, or 280 degrees Fahrenheit sustained for at least 1, 2, 3, 4, 5, 8, 10, 12, 15, 20, or 25 seconds. In some embodiments, a method of thermally processing a packaged food product is included. The method can include placing the packaged food product in an environment with a pressure greater than 0 psig and applying electromagnetic energy to the packaged food product from a first electromagnetic energy source from a first direction. In various embodiments, electromagnetic energy can also be applied from a second, third, fourth, fifth, sixth, etc. energy source from the same direction or from second, third, fourth, fifth, sixth, etc. different directions. The temperature can be raised to at least 50, 60, 70, 80, 90, 100, 110, 120, 130, 140, 150, 160, 170, 180, 190, 200, 210, 220, 230, or 240 degrees Fahrenheit in 600, 540, 480, 420, 360, 330, 300, 270, 240, 210, 180, 150, 140, 130, 120, 110, 100, 90, 80, 70, 60, 50, 40, or 30 seconds or less and at least 30, 40, 50, 60, 70, 80, 90, 95, or 98 percent of the energy required to raise the temperature is provided by electromagnetic energy absorbed by portions of the packaged food product.

### Food Materials and Food Products

Food materials in accordance with embodiments herein can include, but are not limited to, foods of all types as well as drinks of all types, unless used explicitly to the contrary. Food materials herein can include shelf-stable food materials, extended shelf-life food materials, ready-to-eat food materials, chilled food materials, refrigerated food materials, and the like. Shelf-stable food materials/products include those where the material or product is free of pathogens capable of growing in the product at non-refrigerated conditions at which the product is intended to be held during distribution and storage. Food materials/products that can be safely stored at room temperature, or “on the shelf,” are called “shelf stable.”

Food materials herein can include acidified and non-acidified food materials. By way of example, food materials can include those having a pH of below 4.6 as well as food materials having a pH of 4.6 or higher. Food materials herein can include high nutritional density food materials. Food materials herein can include human food materials, pet food materials, geriatric food materials, food materials for at-risk populations, baby food materials, nutraceuticals, and the like. Food materials herein can include, but are not limited to, soups, soups with particulates, sauces, concentrates, condiments, salsas, dips, fruits, vegetables, nut products, grain products, pasta products, food components or ingredients, beverages of all types, dairy products, meat products, fish products, entrees, combinations of any of these, and the like. In some embodiments, food materials herein include those that remain in a flowable state after exposure to thermal energy used for sterilization and/or pasteurization. In some embodiments, food materials herein include those that can be deformed in shape, then thermally treated using electromagnetic waves, and then return to an original or default package shape.

While not intending to be bound by theory, spatial temperature variation can be reduced by using food materials with substantially homogeneous dielectric properties (e.g., dielectric loss factor and dielectric constant). As such, in various embodiments herein, food materials can include those with substantially homogeneous dielectric properties. In some embodiments, at least 70, 80, 90, 95, or 98% of the food material by weight exhibits dielectric properties (at least one of dielectric loss factor and dielectric constant) that vary by less 50, 40, 30, 20, 10, or 5%.

### Packaging

Food packaging for packaged food products herein can include various types of packages and containers. As used herein, the term “food package” shall be synonymous with the term “food container”. Food packages/containers can include many different types including, but not limited to, jars, cans, bottles, bowls, trays, multi-pack packages, bags, sleeves, pouches, and the like. Food packages/containers can be rigid, semi-rigid, semi-flexible, or flexible. In various embodiments the food packages herein can be substantially transparent to microwave energy. In various embodiments portions of food packages herein can be substantially transparent to microwave energy while other portions can absorb or reflect microwave energy.

Many different materials can be used to make food packages/containers herein. Materials can include, but are not limited to, polyesters, polyethylene terephthalate (crystallized or amorphous), polyamide (NYLON), oriented polyamide, bi-oriented polyamide, polycarbonate, polyetherimide, polyolefins such as polypropylene or polyethylene, ethylene vinyl alcohol, various adhesives and the like.

Food packages/containers herein can have many different shapes including, substantially box-like, cup-like, bowl-like, and the like. In accordance with various embodiments herein, the packaging can be selected in order to aid in the uniform application of electromagnetic energy to the food material. By way of example, in some embodiments, the food package can be at least partially toroidal (by virtue of its inherent shape and/or by virtue of being temporarily pressed into such a shape). While not intending to be bound by theory, it is believed that the uniformity of electromagnetic fields received by food materials can be enhanced through the use of a food package that is toroidal or semi-toroidal. Aspects of toroidal packaging are described in U.S. Pat. Appl. No. 62/673,177, the content of which is herein incorporated by reference.

### Electromagnetic Energy Emitting Apparatus

In accordance with various embodiments herein, the microwave/radiofrequency energy emitting apparatus and/or microwave/radiofrequency energy source can provide a high-intensity, highly-uniform electromagnetic energy field. Microwave field intensities herein can be very high and can be characterized as a voltage gradient in free space, e.g., volts per centimeter. In some embodiments herein, the field strength can be greater than 0, 10, 20, 30, 40, 50, 60, 70, 80, 90, 100, 110, 120, 130, 140, 150, 175, 200, 225, 250, 275, 300, 325, 350, 375, 400, 425, 450, or more V/cm (or the field strength can fall within a range between any of the foregoing). Field strength can be measured using various instruments including, but not limited to, a Luxtron Model MEF-1.5 Microwave E-Field Probe, (available from Luxtron Corp., Mountain View, Calif.).

Microwave fields herein can be highly uniform. In some embodiments, microwave fields herein can exhibit variation in field uniformity throughout the area of microwave energy application at a surface of the food material (such as at a top and/or bottom surface of the food material) is less than 40, 30, 25, 20, 15, 10 or 5% (or falling within a range between any of the foregoing), such as if measured by a microwave sensitive diode.

Microwave equipment to produce electromagnetic fields for use with embodiments herein include microwave equipment available from Cober Electronics, Inc. and APV Baker, Inc. (See, e.g., GB 2,193,619A, incorporated herein by reference.) Further microwave equipment is described in U.S. Pat. No. 7,208,710, the content of which is herein incorporated by reference.

In a radiofrequency energy source, an RF generator creates an alternating electric field between two electrodes. The packaged food product can be conveyed between the electrodes where the alternating energy caused polar molecules in the product material to continuously reorient themselves to face opposite poles much like the way bar magnets behave in an alternating magnetic field. The friction resulting from molecular movement causes the material to rapidly heat throughout its entire mass. The amount of heat generated in the food material is determined by the frequency, the square of the applied voltage, dimensions of the component and the dielectric loss factor of the food material. Exemplary radiofrequency source components can include the MACROWAVE Model L-200, or portions thereof, commercially available from the Radio Frequency Company, Millis, Mass.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A method of thermally processing a packaged food product comprising:
placing the packaged food product in a heating chamber, the packaged food product comprising a hermetically sealed package and a food material disposed within the package, the heating chamber having a pressure greater than 0 psig; and
applying electromagnetic energy to the packaged food product from a first electromagnetic energy source from a first direction;
wherein the electromagnetic energy is sufficient to raise the temperature of the food material by at least 50 degrees Fahrenheit with a spatial temperature variation of less than 25 degrees Fahrenheit in less than 120 seconds.

2. The method of claim 1, wherein the electromagnetic energy comprising microwave energy.

3. The method of claim 1, wherein the electromagnetic energy comprising radiofrequency wave energy.

4. The method of claim 1, wherein the temperature of the food material product is raised by at least 50 degrees Fahrenheit in less than 90 seconds.

5. The method of claim 1, wherein the temperature of the food material product is raised by at least 50 degrees Fahrenheit in less than 60 seconds

6. The method of claim 1, wherein the electromagnetic energy is sufficient to raise the temperature of the food material by at least 50 degrees Fahrenheit with a spatial temperature variation of less than 15 degrees Fahrenheit in less than 120 seconds.

7. The method of claim 1, wherein the electromagnetic energy is sufficient to raise the temperature of the food material by at least 50 degrees Fahrenheit with a spatial temperature variation of less than 10 degrees Fahrenheit in less than 120 seconds.

8. The method of claim 1, wherein the electromagnetic energy is applied at least 50 percent of the time during a 180 second period.

9. The method of claim 1, wherein the electromagnetic energy is applied at least 70 percent of the time during a 180 second period.

10. The method of claim 1, wherein the electromagnetic energy is applied at least 90 percent of the time during a 180 second period.

11. The method of claim 1, wherein the spatial temperature variation is less than 10 degrees Fahrenheit.

12. The method of claim 1, further comprising applying electromagnetic energy to the packaged food product from a second electromagnetic energy source from a second direction, wherein the second direction is substantially opposite the first direction.

13. The method of claim 12, wherein the first direction is from the top and the second direction is from the bottom.

14. The method of claim 1, wherein the packaged food is immersed in a fluid within the heating chamber.

15. The method of claim 14, wherein less than 60% of the energy required to raise the temperature of the food material to a preselected temperature is provided through conductive heat transfer from the fluid to the packaged food product.

16. The method of claim 15, wherein the preselected temperature is at least one of
a maximum attained processing temperature;
a required lethality temperature;
a targeted lethality temperature; or
a targeted process temperature.

17. (canceled)

18. The method of claim 14, wherein less than 10% of the energy required to raise the temperature of the food material to a preselected temperature is provided through conductive heat transfer from the fluid to the packaged food product.

19-25. (canceled)

26. The method of claim 1, wherein the total time between the beginning of the application of electromagnetic energy and the beginning of a hold phase is less than 1200 seconds.

27-28. (canceled)

29. A method of thermally processing a packaged food product comprising:
placing the packaged food product in an environment with a pressure greater than 0 psig; and
applying electromagnetic energy to the packaged food product from a first electromagnetic energy source from a first direction;
wherein the electromagnetic energy is applied at least 50 percent of the time during a 180 second period, wherein the average temperature of the food material is increased by at least 100 degrees Fahrenheit and no portion of the food material reaches a temperature exceeding 265 degrees Fahrenheit sustained for at least 5 seconds.

30-32. (canceled)

33. A method of thermally processing a packaged food product comprising:
placing the packaged food product in an environment with a pressure greater than 0 psig; and
applying electromagnetic energy to the packaged food product from a first electromagnetic energy source from a first direction;
wherein the temperature is raised to at least 100 degrees Fahrenheit in 180 seconds or less and at least 40 percent of the energy required to raise the temperature is provided by electromagnetic energy absorbed by portions of the packaged food product.

34-36. (canceled)
